(19) United States
(12) Patent Application Publication
(10) Pub. No.: US 2021/0330570 A1
(43) Pub. Date: Oct. 28, 2021

(54) FILM-FORMING COMPOSITION BASED ON LEGUMINOUS STARCH FOR COSMETIC USE

(71) Applicant: ROQUETTE FRERES, Lestrem (FR)

(72) Inventors: Léon MENTINK, Lille (FR); Grégory LE BIHAN, Cambrin (FR); Jérémie BIRRAUX, Paris (FR)

(21) Appl. No.: 17/309,692

(22) PCT Filed: Dec. 13, 2019
(86) PCT No.: PCT/FR2019/053059

§ 371 Date: Jun. 15, 2021

(30) Foreign Application Priority Data
Dec. 17, 2018 (FR) 18 73080

(51) Int. Cl. A61K 8/73; A61K 8/34; A61Q 19/00; A61Q 5/00

(52) U.S. Cl. CPC A61K 8/732; A61Q 5/00; A61Q 19/00; A61K 8/345

(57) ABSTRACT

The present invention relates to novel film-forming compositions having good mechanical strength during application and also an ability to be easily eliminated or removed by water washing. The invention also relates to cosmetic care or make-up compositions comprising a film-forming composition as well as to their non-therapeutic uses.

### FIELD OF THE INVENTION

The invention relates to a film-forming composition, a cosmetic care or makeup composition containing it, a makeup and care cosmetic method and various cosmetic uses.

### BACKGROUND ART

Film-forming agents are known in cosmetics for their ability to produce a continuous film on the skin, hair or nails.

Typically, capillary cosmetic compositions contain film-forming polymers to produce a coating film on the hair. These properties are used in particular to enhance the hairstyle.

Applied to the skin, film-forming agents oppose evaporation of water and thus reduce water losses. These agents provide deep and lasting hydration to the skin. They can also help protect the skin, for example by having a barrier effect against pollution, including pollution by airborne microparticles, and ultraviolet rays. In makeup and typically in mascaras, film-forming agents significantly increase eyelash curl and lengthening. In solar protection, film-forming agents can form films that block UV radiation, or reduce the amount of radiation that reaches the skin.

Polymers of natural origin have also been used in cosmetics for their powerful biomechanical and film-forming properties conferring second protective skin efficiency. Polymers of natural origin are those extracted or obtained from renewable natural resources, i.e. plants, such as starches and their derivatives, celluloses and their derivatives, gums from plant seeds or exudate, gums extracted from algae, either from the fermentation of micro-organisms such as fungi, bacteria, such as xanthan gums, carrageenans, etc.

Cosmetic compositions comprising mixtures of ingredients that provide elasticity to the cosmetic mask-type product applied to the surface of the skin naturally stressed by deformations have been described in document WO 2013/167835. Typically, cosmetic compositions containing a mixture suitable for forming an elastic film consisting of at least one starch hydrolyzate, at least one polyol, at least one polymer selected from polyvinyl alcohols, vinylpyrrolidone polymers and copolymers, and latex, and possibly water are described.

These film-forming agents or compositions still contain a significant share of synthetic polymers, and are difficult to rinse or remove. Furthermore, it is difficult to obtain compositions with good mechanical holding during application.

It is thus necessary to develop a cosmetic film-forming composition of natural origin which presents a good compromise between good mechanical hold during application and a good ability to be eliminated after application, for example by washing.

### BRIEF DESCRIPTION OF THE INVENTION

It is to the Applicant's credit to have developed new film-forming compositions for cosmetic use of natural origin with good mechanical holding during application as well as a good ability to eliminate by simple rinsing or washing.

These film-forming compositions also provide keratinous materials with excellent properties in terms of improved skin tone, even skin tone, homogeneous skin tone, obtaining a matte skin tone, skin smoothing, protecting the sensitized areas of a hair fiber, smoothing the scales of hair fibers, sheathing of hair fibers, curling and lengthening of eyelashes, and resistance to mechanical impacts. Another effect is the barrier effect to external aggression such as airborne micro pollutants, UV rays, oxidants, by limiting or preventing their migration to dermis or keratinous materials.

The present invention also relates to cosmetic compositions for protection, care or makeup comprising a film-forming composition of natural origin and a method for makeup or care of keratinous materials comprising the application of at least one cosmetic composition according to the present invention on said keratinous materials.

### DISCLOSURE OF THE INVENTION

A first object of the invention thus relates to a film-forming composition, comprising:
1% to 20% of at least one hydrolyzed and alkylated leguminous starch,
1% to 20% of at least one polyol, and
1% to 10% of at least one carrageenan,
the percentages being expressed by weight relative to the total weight of the film-forming composition.

Generally speaking, “film-forming composition” is understood to mean a composition comprising at least one polymer (film-forming agent), said polymer being capable of forming a substantially continuous film in the presence of a solvent, in particular water. For the purposes of the present invention, the film-forming compositions are in particular film-forming compositions of natural origin based on starch, in the sense that starch is used as the main film-forming agent.

“Leguminous” means any plant belonging to the families of the cesalpiniaceae, mimosaceae or papilionaceae, and in particular any plant belonging to the family of the papilionaceae such as, for example, peas, green beans, fava beans, horse beans, lentils, or lupins.

According to a preferred embodiment, the leguminous starch is a pea starch.

The leguminous starch according to the invention is obtained from starch with an amylose content of 25% to 45%, preferably from 30% to 45%, and preferably from 35% to 40%, where the percentages are expressed in dry weight relative to the dry weight of leguminous starch, and determined before any subsequent treatment such as hydrolysis and/or alkylation of said starch.

The leguminous starch according to the invention is a modified starch. It is a hydrolyzed and alkylated starch, preferably hydroxypropyl.

Thus, according to a preferred embodiment, the starch according to the invention is a hydrolyzed and hydroxypropylated leguminous starch.

“Hydroxypropylated starch” is understood to mean, for the purpose of the present invention, starch substituted by hydroxypropyl groups by any technique known to the person skilled in the art, for example by etherification reaction with propylene oxide, in particular presenting a hydroxypropyl group content between 0.1 and 20% by dry weight relative to the dry weight of hydroxypropylated starch, preferably between 1 and 10%, more preferably between 5 and 9%, and in particular close to 7%. In particular, this content is determined by proton Nuclear Magnetic Resonance spectrometry, in particular according to the EN ISO 11543:2002 F standard.

“Hydrolyzed starch” is understood to mean, for the purpose of the present invention, a starch which has undergone a hydrolysis operation, i.e. an operation to reduce its mean molecular mass. The person skilled in the art knows how to obtain such starches, for example by chemical treatments such as oxidation and acid treatments, or also by enzymatic treatments. The person skilled in the art will naturally adjust the fluidification level of the starch based on the viscosity desired for the film-forming composition.

Hydrolyzed and alkylated leguminous starch according to the invention may also comprise one or more other physical and/or chemical modifications, provided that said modifications do not interfere with the desired properties of said starch within the film-forming composition according to the invention. An example of chemical modification is cross-linking.

According to a particular embodiment, hydrolyzed and alkylated starch of the film-forming composition according to the invention may further have other modifications, and may for example have undergone physical treatments, particularly selected from the known gelatinization, pre-gelatinization, extrusion, atomization or drying operations, microwave or ultrasonic processing, plasticizing or granulation treatment operations.

In particular, starch after alkylation and hydrolysis will preferably be non-granular. It will be advantageously rendered soluble in water by any known technique so that the film-forming composition presents very good film-forming properties.

Thus, according to an advantageous embodiment, starch according to the invention is characterized in that it is rendered soluble. It can be made soluble by any technique known to the person skilled in the art, including by heat and/or mechanical treatment, for example by cooking in aqueous media, possibly followed by a drying step when obtaining a powder product is preferred. The operation to make the starch soluble may occur before the addition of the starch into the film-forming composition, in an undifferentiated manner before or after the alkylation and/or hydrolysis of the starch, or after its addition into the film-forming composition, for example by cooking the film-forming composition at the time of its implementation.

Hydrolyzed and alkylated starch according to the invention has a preferred mean molecular weight of 1 to 2,000 kDa, preferably 10 to 1,000 kDa, more preferably 20 to 1, 000 kDa, and even more preferably 100 to 1,000 kDa. For example, the molecular weight can range from 200 to 800 kDa, from 200 to 500 kDa, from 200 to 400 kDa, or from 200 to 300 kDa. The mean molecular weight is determined by HPSEC-MALLS (high-performance size exclusion chromatography coupled in-line with multi-angle laser light scattering detection).

Hydrolyzed and hydroxypropyl starches suitable for the film-forming composition according to the invention are commercially available and are offered by the Applicant under the brand name Beauty by Roquette® ST720.

The film-forming composition comprises 1% to 20%, preferably 1% to 15%, and particularly 1% to 10% by weight of hydrolyzed and alkylated leguminous starch. The percentages by weight being expressed relative to the total weight of the film-forming composition.

The film-forming composition according to the invention also comprises one or more polyols. Polyol is used in the film-forming composition as a plasticizing agent.

Any polyol known to the person skilled in the art can be used in the film-forming composition according to the invention.

According to a particular embodiment, polyol is selected from the group consisting of maltitol, mannitol, xylitol, erythritol, sorbitol, isosorbide, glycerol or glycerin, glucose, sucrose, polydextrose, hydrogenated glucose syrups, dextrins, maltodextrins, glucose syrups, and mixtures thereof. The polyol is preferably selected from glycerol and sorbitol.

The film-forming composition comprises 1% to 20%, preferably 1% to 15%, and particularly 1% to 10% by weight of polyols, with the percentages by weight expressed relative to the total weight of the film-forming composition.

The film-forming composition according to the invention also comprises at least one carrageenan. Carrageenan is used as a thickening agent within the film-forming composition.

According to a preferred embodiment, carrageenan is selected from carrageenan iota, carrageenan lambda, carrageenan kappa, and their mixtures. Carrageenan is present in the film-forming composition according to a quantity between 1 and 10%, preferably 1 to 7%, and even more preferably, 1% to 5% by weight. The percentages by weight being expressed in relation to the total weight of the film-forming composition.

According to a particular embodiment, the film-forming composition may further comprise a buffer agent selected from calcium or potassium salts, such as calcium chloride, potassium chloride, monobasic potassium phosphate and dibasic calcium phosphate.

According to a particular embodiment, the film-forming composition according to the invention is an aqueous composition. According to this particular embodiment, it has a dry matter content of 1 to 50% by weight, preferably 2 to 40% by weight, and more preferably 5 to 25% by weight.

According to a particular embodiment, the film-forming composition may further comprise:
other film-forming agents than hydrolyzed and alkylated starch used in accordance with the invention, for example polymers of natural origin such as hydroxypropyl methyl cellulose (HPMC), hydroxypropyl cellulose (HPC), or synthetic polymers such as polyvinyl alcohol (PVA),—and/or
plasticizers other than polyols according to the invention, such as polyethylene glycol, triethylcitrate, polysorbate, or waxes such as Carnauba wax, or hydrogenated castor oil.

In order to facilitate the implementation, however, it is preferable to minimize the amount of raw materials to be used. As a result, and preferentially, hydrolyzed and alkylated starch and polyol represent in the film-forming composition according to the invention the most, and preferably all, of the film-forming agents and plasticizers respectively.

Advantageously, the quantities of the various compounds of the film-forming composition according to the invention may also be expressed as a function of the total dry matter of said film-forming composition. In fact, it is the ratio between the film-forming agent and the plasticizing agent that determines in particular the plasticity and thus the quality of the film formed.

Thus, the film-forming composition according to the invention comprises:
20 to 60%, preferably 30% to 50% of at least one hydrolyzed and alkylated leguminous starch,
20 to 60%, preferably 30% to 50%, of at least one polyol, and
5 to 25%, preferably from 10% to 20%, of at least one carrageenan.
The percentages are expressed in dry weight relative to the total dry weight of the film-forming composition.

According to an advantageous embodiment, hydrolyzed and alkylated starch, polyol and carrageenan together account for between 20% and 100% by dry weight relative to the total dry weight of the film-forming composition, preferably between 30% and 99%, more preferably between 50 and 99%, and even more preferably between 95 and 99% by dry weight relative to the total dry weight of the film-forming composition.

According to a particular embodiment, the film-forming composition contains:
20 to 60%, preferably 30% to 50% of one hydrolyzed and alkylated leguminous starch,
20 to 60%, preferably 30% to 50%, of at least one polyol, and
5 to 25%, preferably from 10% to 20%, of one carrageenan,
optionally from 0.01 to 0.5% of a buffer agent selected from calcium or potassium salts, preferably chosen among calcium chloride, potassium chloride, and monobasic potassium phosphate,
optionally from 0 to 15% of another ingredient, preferably from 0 to 5%, more preferably from 0 to 3%, and in particular from 0 to 1%. 0

The percentages are expressed in dry weight relative to the total dry weight of the composition, with their total sum being 100%.

The film-forming compositions of the invention may indeed contain other ingredients provided that they do not interfere with the desired properties of said film-forming compositions. These other ingredients may be, for example, surfactants, opaque fillers, preservatives, antimicrobial agents, sweeteners, flavoring substances, dyes, optical brighteners or even lubricants.

The present invention also relates to the cosmetic use of the film-forming composition according to the present invention as previously described.

Also, the present invention relates to the non-therapeutic use of a film-forming composition according to the present invention to confer to keratinous materials at least one property chosen among improved skin tone, even skin tone, homogeneous skin tone, obtaining a matte skin tone, barrier effect, skin smoothing, protecting the sensitized areas of a hair fiber, smoothing the scales of hair fibers, sheathing of hair fibers, curling and lengthening of eyelashes, and resistance to mechanical impacts.

Indeed, by its properties, the film-forming composition in the sense of the present invention will allow to even out the skin tone, make it uniform, homogeneous and matte. The film-forming properties of the composition according to the present invention as previously described will allow, when the composition according to the present invention is applied on keratinous materials, preferably on the skin of the face or body, to smoothen the skin, mask imperfections in order to obtain a more evened out, uniform, homogeneous and matte skin tone.

The film-forming composition within the meaning of the present invention will protect the sensitized areas of a hair fiber, smooth the scales of hair fibers and sheath hair fibers. These properties are obtained by forming a film on the surface of the hair fiber.

The film-forming composition within the meaning of the present invention allows the curling and lengthening of keratinous fibers, preferably eyelashes.

The film-forming composition within the meaning of the present invention will confer a barrier effect on keratinous materials. Indeed, the film-forming composition according to the present invention will protect keratinous materials from external attacks such as airborne micropollutants, UV rays, oxidizing them, by limiting or preventing their migration to the dermis or core of the hair fiber. Thus, “barrier effect” is understood to mean the protection of keratinous materials against external aggression within the meaning of the present invention.

The film-forming composition is as previously defined. The present invention also relates to a cosmetic composition for makeup, care or protection of keratinous materials comprising, in a physiologically acceptable medium, a film-forming composition comprising at least one hydrolyzed and alkylated leguminous starch, at least one polyol, and at least one carrageenan.

The film-forming composition, including hydrolyzed and alkylated leguminous starches, polyols, and carrageenans, are as previously defined.

The cosmetic composition may further comprise a buffer agent as previously defined.

According to one embodiment, the film-forming composition present in the cosmetic composition for makeup, care or protection of keratinous materials comprises:
1% to 20% of at least one hydrolyzed and alkylated leguminous starch, preferably of at least one hydrolyzed and hydroxypropyl pea starch,
1% to 20% of at least one polyol, preferably selected from sorbitol and glycerol, and
1% to 10% of at least one carrageenan, the percentages being expressed by weight relative to the total weight of the film-forming composition.

The physiologically acceptable medium is preferably cosmetically or dermatologically acceptable. “Physiologically acceptable medium” or “cosmetically acceptable” or “dermatologically acceptable” is understood to mean, for the purpose of the present invention, a medium which does not present deleterious side effects and in particular does not produce redness, inflammation, heating, tightness or tingling unacceptable for a user of cosmetic products. The medium is thus compatible with the keratinous materials of human beings.

Typically, keratinous materials will be skin, mucous membranes, scalp or hair, hair system, eyelashes, eyebrows, and nails.

According to one embodiment, the film-forming composition within the meaning of the present invention shall confer on the cosmetic makeup or care composition at least one property selected from evening out, uniformity, homogeneity of the skin tone or obtaining a matte skin tone. The film-forming properties of the composition will allow, when the composition is applied to keratinous materials, preferably on the face or body, to smooth the skin, to mask imperfections in order to obtain a more evened out, uniform, homogeneous and matte skin tone.

According to another embodiment, the film-forming composition within the meaning of the present invention confers on the makeup or care cosmetic composition properties of protection of sensitized areas of a hair fiber, smoothing of scales of hair fibers, and sheathing of hair fibers. These properties are obtained by forming a film on the surface of the hair fiber.

According to another embodiment, the film-forming composition within the meaning of the present invention will confer on the makeup or care cosmetic composition properties of curling and lengthening of keratinous fibers, preferably of eyelashes.

According to another embodiment, the film-forming composition within the meaning of the present invention confers a barrier effect on keratinous materials. Barrier effect is understood to mean the protection provided by the present film-forming composition to keratinous materials, by preventing or limiting the migration of agents responsible for external aggression such as atmospheric micropollutants, atmospheric oxidizing materials or compounds, or sunlight, such as ultraviolet rays, to the dermis or core of the hair fiber.

According to another embodiment, the film-forming composition within the meaning of the present invention confers on the cosmetic makeup or care composition properties of resistance to mechanical impacts, by forming a film on the surface of the keratinous material, typically on the surface of a nail.

Thus, the present invention relates also to non-therapeutic use of a cosmetic composition for makeup, protection or care of keratinous materials as previously defined intended to confer to keratinous materials at least one property chosen among improved skin tone, even skin tone, homogeneous skin tone, obtaining a matte skin tone, a barrier effect, skin smoothing, protecting sensitized areas of a hair fiber, smoothing the scales of hair fibers, sheathing of hair fibers, curling and lengthening of eyelashes, and resistance to mechanical impacts

The cosmetic composition for makeup, protection or care according to the invention may come in all dosage forms used in the cosmetic field, normally used for topical application such as emulsions (in particular cream or milk), gels, creams, solutions, dispersions of lotion or serum type, emulsions of liquid or semiliquid consistency obtained by dispersion of a fatty phase in an aqueous phase (oil-in-water emulsion, O/W) or vice versa (water-in-oil emulsion, W/O), suspensions, lotions, microemulsions, microcapsules, vesicular microparticles or dispersions of ionic and/or non-ionic type, polishes, body oils, masks, creams, ointments, and concentrated solutions. These dosage forms are obtained according to the usual methods known to the person skilled in the art. These dosage forms further contain cosmetologically typical and acceptable excipients and supports.

As an illustration, the physiologically acceptable medium may comprise a fatty phase and/or an aqueous phase.

### Fatty Phase

The fatty phase may contain at least one oil.

For the purposes of the present invention, “oil” is understood to mean a compound which is liquid at room temperature (25° C.) and which, when it is introduced at an amount of at least 1% by weight into the water at 25° C., is not at all soluble in the water, or soluble to an extent of less than 10% by weight, relative to the weight of oil introduced into the water.

The liquid fatty phase advantageously comprises one or more nonvolatile oils which afford an emollient effect on the skin. Mention may be made of fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, triacetin, tricaprin, caprylic/capric acid triglycerides, coco caprate and caprylate mixture, benzoates of C12 to C15 alcohols, glycol esters such as butylene glycol cocoate, glyceryl triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, vegetable oils such as avocado oil, camellia oil, hazelnut oil, tsubaki oil, cashew nut oil, argan oil, soybean oil, grape seed oil, sesame oil, corn oil, wheatgerm oil, rapeseed oil, sunflower oil, cottonseed oil, jojoba oil, peanut oil, olive oil and mixtures thereof, vegetable butters such as shea butter, camellia butter.

These oils may also be hydrocarbon or silicone type oils, such as paraffin oils, squalane, petroleum jelly, dimethyl siloxanes and mixtures thereof.

The liquid fatty phase may also comprise volatile oils. Volatile oil is intended to mean an oil which is capable of evaporating from the skin in less than one hour at room temperature and atmospheric pressure. The volatile oils may for example be selected from silicone oils or short-chain fatty acid triglycerides in order to reduce the greasy feel.

The fatty phase of the composition according to the invention may further advantageously comprise at least one fatty phase structuring agent such as a lipophilic gelling agent, a wax and/or a pasty compound.

“Wax” is intended to mean a fatty substance having reversible liquid-solid state change and having a melting point of greater than 30° C. and generally less than 90° C., which is liquid under the preparation conditions of the composition and which has anisotropic crystalline organization in the solid state. The waxes used according to the invention may consist of polar or apolar waxes or a mixture of these two. “Apolar” is intended to mean a wax containing only carbon, hydrogen and/or phosphorus atoms, and in particular a hydrocarbon.

The polar waxes may be selected from animal waxes, vegetable waxes, and synthetic or silicone waxes containing polar groups such as esters. Mention may thus be made of carnauba wax, candellila wax, beeswax (Cera alba), Chinese insect wax (Ericerus pela), Japan wax, sumac wax, montan wax, triesters of C8-C20 acids and glycerin, such as glyceryl tribehenate, acetylated glycol stearate, sold especially by VEVY under the trade name CETACENE, and mixtures thereof. These waxes may in particular be used in predispersed form in an oil, as is the case of the mixture of candelilla wax and jojoba seed oil. “Pasty compound” is understood to mean lipophilic fatty substances which, like waxes, are capable of undergoing a reversible liquid-solid state change and which have, in the solid state, anisotropic crystalline organization, but which differ from waxes in that they contain, at a temperature of 23° C., a liquid fraction and a solid fraction.

### Aqueous Phase

The cosmetic composition according to the invention may further comprise an aqueous phase comprising water and optionally one or more water-miscible organic solvents and/or one or more rheology agents.

The rheology agent may especially be an agent for thickening the aqueous phase, a gelling agent or a suspending agent, for example plant-derived gums such as gum arabic, konjac gum, guar gum or derivatives thereof; gums extracted from algae such as alginates; gums resulting from microbial fermentation such as xanthans, mannans, scleroglucans or derivatives thereof, cellulose and derivatives thereof such as carboxymethylcellulose or hydroxyethylcellulose; starch and derivatives thereof such as in particular modified starches and derivatives thereof, especially acetylated, carboxymethylated, octenylsuccinates or hydroxypropylated; synthetic polymers such as polyacrylic acids or carbomers.

### Surfactants

The composition according to the invention may also comprise one or more surfactants, preferably selected from water-in-oil (W/O) or oil-in-water (O/W) emulsifiers.

The oil-in-water (O/W) emulsifier may especially be selected from sorbitan esters which could be polyethoxylated, glycerol fatty acid esters, sucrose fatty acid esters or polyesters, polyethylene glycol fatty acid esters, polyether-modified polysiloxanes, polyethyleneglycol fatty alcohol ethers, alkyl polyglycosides and hydrogenated lecithin, this list being non-limiting.

The water-in-oil (W/O) emulsifier may be selected from non-ethoxylated polyol fatty esters, and especially from non-ethoxylated fatty esters of glycerol, of polyglycerols, of sorbitol, of sorbitan, of anhydrohexitols, such as in particular isosorbide, mannitol, xylitol, erythritol, maltitol, sucrose, glucose, polydextrose, hydrogenated glucose syrups, dextrins and hydrolyzed starches.

The W/O emulsifier may be selected from non-ethoxylated polyol fatty esters obtained from fatty acids or by transesterification from oil or oil mixtures. The fatty acids used comprise from 8 to 22 carbon atoms, preferably from 10 to 18 carbon atoms, and in particular from 12 to 18 carbon atoms. These acids may be linear or branched, saturated or unsaturated and have one or more lateral hydroxyl functions. The oils may be saturated or unsaturated, from liquid to solid at room temperature, and optionally have hydroxyl functions, preferably an iodine value of between 1 and 145 and in particular from 5 to 105.

The W/O emulsifier can be advantageously selected from emulsifying systems consisting of a cyclodextrin and a natural water-in-oil emulsifier, such as the emulsifying system marketed by Roquette Freres under the name Beauty by Roquette® DS146.

### Dyestuff

The composition according to the invention may further comprise at least one dyestuff selected from water-soluble or liposoluble dyes, fillers with the effect of coloring and/or opacifying the composition and/or coloring the lips, such as pigments, nacres, lakes (water-soluble dyes adsorbed on an inert mineral support) and mixtures thereof. These dyestuffs may be optionally surface-treated by a hydrophobic agent such as silanes, silicones, fatty acid soaps, C9-C15 fluoroalcohol phosphates, acrylate/dimethicone copolymers, C9-C15 fluoroalcohol phosphate/silicon mixed copolymers, lecithins, carnauba wax, polyethylene, chitosan and optionally amino acids which could be acylated such as lauroyl lysine, disodium stearoyl glutamate and aluminum acyl glutamate. The pigments may be mineral or organic, natural or synthetic. Examples of pigments are especially iron, titanium or zinc oxides, and also composite pigments and goniochromatic, pearlescent, interference, photochromic or thermochromic pigments, with this list being non-limiting.

### Fillers

The cosmetic composition according to the invention may further contain at least one filler. This term is intended to mean particles of any form (especially spherical or lamellar), mineral or organic, which are insoluble in the composition. Examples of fillers are talc, mica, silica, kaolin, boron nitride, starch, starch modified with octenylsuccinic anhydride, polyamides, silicone resins, silicone elastomer powders and acrylic polymer powders, in particular powders of poly(methyl methacrylate) or powders of styrene acrylate copolymer (Sunsphere Powders from Dow).

### Cosmetic Applications

According to an embodiment, the cosmetic composition for care is in the form of a mask. The present invention thus relates to a cosmetic composition for mask-type care comprising in a physiologically acceptable medium a film-forming composition comprising at least one hydrolyzed and alkylated leguminous starch, at least one polyol, at least one carrageenan.

According to a notion well known to a person skilled in the art, a mask-type composition is understood to mean a cosmetic composition formulated to be applied on keratinous materials, preferably the skin in a more or less thick layer. Mask-type compositions are intended to be left on keratinous materials during a set time, for example from a few minutes to several tens of minutes. Mask-type cosmetic compositions may be left on keratinous materials for an entire night for a prolonged effect. At the end of the prescribed time, the mask composition may be rinsed with water to detach or remove it.

It is to the Applicant's credit to have developed a cosmetic composition of the mask type, thanks to the film-forming composition according to the present invention, presenting excellent mechanical properties, including good mechanical hold, and a good ability to be eliminated quickly and easily after application by washing with water.

According to an embodiment, the present invention also relates to a cosmetic composition for makeup, protection or care of keratinous materials comprising a film-forming composition and at least one cosmetically active ingredient, said film-forming composition comprising at least one hydrolyzed and alkylated leguminous starch, at least one polyol, at least one carrageenan.

A cosmetically active ingredient or cosmetically active substance is understood to mean any ingredient or substance having an anti-aging activity, a hydrating activity, a firming effect, a depigmenting activity, a skin brightening activity, a matte effect, a soothing effect, a microcirculation stimulating activity, a seboregulating activity, an epidermal desquamation activity, an ingredient or substance intended to clean or purify the skin, and a firming effect.

Thus, in addition to the film-forming composition previously described, the cosmetic composition according to the invention may, moreover, comprise one or more hydrophilic or lipophilic active ingredients. In particular, additional active ingredients may be selected from the group consisting of vitamins, antioxidants, anti-aging agents, moisturizing agents, antipollution agents, keratolytic agents, astringents, anti-inflammatory agents, whitening agents and agents promoting microcirculation, seboregulators, epidermal desquamation agents, or agents with a firming effect.

Preferably, in addition to the film-forming composition previously described, the cosmetic composition according to the invention may comprise at least one active ingredient selected among anti-aging agents, epidermal desquamation agents, seboregulating agents, agents with a firming effect and mixtures thereof, for example, gluconolactone or hydroxypropyl cyclodextrin.

Any technique for incorporating a cosmetically active ingredient into the film-forming composition may be used by the person skilled in the art.

Typically, in the case of a mask type cosmetic composition, the active ingredient may be incorporated into the film-forming composition in order to obtain the desired cosmetic effect when applying the mask type cosmetic composition to keratinous materials, preferably skin and hair.

Gluconolactone is an active ingredient known for its seboregulating, anti-aging and epidermal desquamation or “peeling” properties.

Typically, gluconolactone may be incorporated into the film-forming composition in order to confer seboregulating, anti-aging and epidermal desquamation properties on the resulting cosmetic composition.

Typically, a hydroxypropyl cyclodextrin may be incorporated into the film-forming composition in order to confer a firming effect and anti-wrinkle properties on the resulting cosmetic composition obtained

The present invention also relates to a method of obtaining a mask-type cosmetic film from a film-forming composition characterized in that it comprises the steps of:
supplying of a film-forming composition;
heating of said composition at a temperature between 40 and 90, preferably 70 and 80° C.,
drying at a temperature between 20 and 50° C. and between 10 and 50% relative humidity.

The film-forming composition provided is as previously described.

The present invention also relates to a method for makeup, care or protection of keratinous materials comprising the application of at least one cosmetic composition according to the present invention to said keratinous materials;

The present invention also relates to a method for makeup, care or protection of keratinous materials comprising the application of at least one film-forming composition according to the present invention on said keratinous materials.

The invention also relates to the use of a film-forming composition according to the present invention for the formation of an elastic film.

The non-therapeutic use of the cosmetic composition in accordance with the invention comprises all body and skin care, such as masks, including products for improving the appearance of the skin. Non-therapeutic use in accordance with the invention also comprise makeup products such as nail polish, mascaras, lipsticks, or hair products such as shampoos, conditioners, care products, masks, restorative and/or protective hair oils. Advantageously, the film-forming composition according to the present invention may be incorporated into all body and skin care products as well as in all the aforementioned makeup products.

The non-therapeutic use of the cosmetic composition in accordance with the invention also comprises all skin protection by barrier effect or second skin effect, against solar radiation, especially against ultraviolet rays.

The invention is also described in the examples below, which are purely illustrative and in no way limit the scope of the present invention.

### EXAMPLES

Three film-forming compositions according to the invention are prepared according to the compositions given in Tables 1 to 3.

TABLE 1

| | | | | % total | % mass/ |
|---|---|---|---|---|---|
| No | Ingredients | Trade name | Supplier | mass | dry mass |
| 1 | Water | water | / | Qsp 100% | / |
| 2 | Glycerin | Glycerin | Cooper | 6.00 | 44.4 |
| 3 | Lambda | Prodhygel | Prod'Hyg | 1.50 | 11.1 |
| | carrageenan | CG | | | |
| 4 | Hydrolyzed and | Beauté by | Roquette | 6.00 | 44.4 |
| | hydroxypropyl | Roquette ® | Frères | | |
| | pea starch | ST 720 | | | |
| 5 | Calcium | Calcium | Cooper | 0.02 | 0.1 |
| | chloride | chloride | | | |

Table 1 corresponds to the film-forming composition A.

TABLE 2

| | | | | % total | % mass/ |
|---|---|---|---|---|---|
| No | Ingredient | Trade name | Supplier | mass | dry mass |
| 1 | Water | water | / | Qsp 100% | / |
| 2 | Glycerol | Glycerol | Cooper | 8,935 | 40 |
| 3 | Iota | Viscarin | Soliance | 4,070 | 18.2 |
| | carrageenan | | | | |
| 4 | Hydrolyzed and | Beauté by | Roquette | 8,935 | 40 |
| | hydroxypropyl | Roquette ® | Frères | | |
| | pea starch | ST 720 | | | |
| 5 | Dibasic calcium | | Cooper | 0,390 | 1.8 |
| | phosphate | | | | |

Table 2 corresponds to the film-forming composition B.

TABLE 3

| | | | | % total | % mass/ |
|---|---|---|---|---|---|
| No | Ingredient | Trade name | Supplier | mass | dry mass |
| 1 | Water | water | / | Qsp 100% | / |
| 2 | Sorbitol | | Cooper | 6.00 | 47.9 |
| 3 | Lambda | Prodhygel | Prod'Hyg | 0.75 | 5.9 |
| | carrageenan | CG | | | |
| 4 | Iota | Viscarin | Soliance | 0.75 | 5.9 |
| | carrageenan | | | | |
| 5 | Hydrolyzed and | Beauté by | Roquette | 5.00 | 39.9 |
| | hydroxypropyl | Roquette ® | Frères | | |
| | pea starch | ST 720 | | | |
| 6 | Calcium | Calcium | Cooper | 0.02 | 0.16 |
| | chloride | chloride | | | |

Table 3 corresponds to the film-forming composition C.

The protocol for preparing the compositions A, B and C is as follows:

A water bath is heated to a temperature of 75° C.

All ingredients are added one at a time waiting for complete dissolution of one ingredient before adding the next, in numerical order from Tables 1 to 3.

The temperature of the solution obtained in the previous step is adjusted to a temperature of 72° C.

### Example 2

The film-forming compositions A, B and C are used to prepare films for application on the skin. To do this, for each film-forming composition, the homogeneous solution obtained in example 1 is poured on an appropriate flat glass support, and then one waits a few minutes for the solution to gel. Then it is dried in an oven at 40° C. under the 30% relative humidity for at least 24 hours. At the end of this protocol, a film is obtained with sufficient mechanical strength to be applied to the skin and with sufficient solubility in water to be dissolved by washing with water.

### Example 3

An anti-aging care mask, formed with the film-forming composition according to the invention, was prepared according to the formula in Table 4.

TABLE 4

| | | | | % total | % mass/ |
|---|---|---|---|---|---|
| No | Ingredient | Trade name | Supplier | mass | dry mass |
| 1 | Water | water | / | Qsp 100% | / |
| 2 | Gluconolactone | Beauté by | Roquette | 0.15 | 1.1 |
| | | Roquette ® | Frères | | |
| | | GA290 | | | |
| 3 | Hydroxypropyl | Beauté by | Roquette | 1 | 7.3 |
| | cyclodextrin | Roquette ® | Frères | | |
| | | CD110 | | | |
| 4 | Glycerin | Glycerin | Cooper | 6.00 | 43.9 |
| 5 | Lambda | Prodhygel | Prod'Hyg | 1.50 | 11 |
| | carrageenan | CG | | | |
| 6 | Hydrolyzed and | Beauté by | Roquette | 5.00 | 36.6 |
| | hydroxypropyl | Roquette ® | Frères | | |
| | pea starch | ST 720 | | | |
| 7 | Calcium | Calcium | Cooper | 0.02 | 0.15 |
| | chloride | chloride | | | |
| 8 | Benzoic acid | Solbrol BZ | Lanxess | 0.10 | / |
| | (preservative) | | | | |

To prepare the mask, the gluconolactone is completely dissolved in the volume of water required at 45° C. under stirring. Glycerin, lambda carrageenan, and hydrolyzed and hydroxypropyl pea starch are then added successively, waiting for complete dissolution between each addition at 45° C. It is next heated to 80° C. and calcium chloride is added, and the mixture is stirred until complete dissolution.

The previously prepared mask composition is then poured onto a flat glass support to obtain a thickness of about 3 mm. Then it is dried in an oven at 40° C. under 30% relative humidity for at least 24 hours.

This results in a film with good mechanical strength. It is applied to the skin for at least one night. There is a firming effect on the skin. After removing the mask by washing the skin with water, the skin is softer and appears to be firmed.

1. A film-forming composition comprising
1% to 20% of at least one hydrolyzed and alkylated leguminous starch,
1% to 20% of at least one polyol, and
1% to 10% of at least one carrageenan,
with the percentages expressed by weight relative to the total weight of the film-forming composition.

2. Composition according to claim 1, wherein said leguminous starch is hydrolyzed and hydroxypropylated starch.

3. Composition according to claim 2, wherein content of hydroxypropyl groups is between 0.1 and 20% by dry weight relative to the dry weight of hydroxypropyl starch, preferably between 1 and 10%.

4. Composition according to claim 1, wherein at least one leguminous starch is a pea starch.

5. Composition according to claim 1, wherein at least one polyol is selected from the group consisting of maltitol, mannitol, xylitol, erythritol, sorbitol, glycerol, glucose, sucrose, polydextrose, hydrogenated glucose syrups, dextrins, maltodextrins, glucose syrups, and mixtures thereof.

6. Composition according to claim 1, wherein at least one carrageenan is selected from iota carrageenan, lambda carrageenan, kappa carrageenan, and mixtures thereof.

7. Cosmetic composition for makeup, protection or care of keratinous materials comprising, in a physiologically acceptable medium, a film-forming composition comprising at least one hydrolyzed and alkylated leguminous starch, at least one polyol, and at least one carrageenan.

8. Cosmetic composition according to claim 7, wherein said film-forming composition comprises:
1% to 20% of at least one hydrolyzed and alkylated leguminous starch,
1% to 20% of at least one polyol, and
1% to 10% of at least one carrageenan,
with the percentages expressed in weight relative to the total weight of the film-forming composition.

9. Process of makeup, protection or care of keratinous materials comprising the application of at least one cosmetic composition according to claim 7 on said keratinous materials.

10. Non-therapeutic use of a film-forming composition according to claim 1 intended to confer to keratinous materials at least one property chosen among improved skin tone, even skin tone, homogeneous skin tone, obtaining a matte skin tone, barrier effect, skin smoothing, protecting the sensitized areas of a hair fiber, smoothing the scales of hair fibers, sheathing of hair fibers, curling and lengthening of eyelashes, and resistance to mechanical impacts.
